(19) United States
(12) Patent Application Publication
(10) Pub. No.: US 2026/0335363 A1
(43) Pub. Date: Oct. 29, 2026

(54) METHODS, KITS, AND COMPOSITIONS FOR TREATING STRESS URINARY INCONTINENCE AND/OR OVERACTIVE BLADDER

(71) Applicant: THOMAS JEFFERSON UNIVERSITY, Philadelphia, PA (US)

(72) Inventor: Karolynn Teresa Echols, Philadelphia, PA (US)

(21) Appl. No.: 19/104,812

(22) PCT Filed: Aug. 21, 2023
(86) PCT No.: PCT/US23/30725

§ 371 Date: Feb. 19, 2025

Related U.S. Application Data
(60) Provisional application No. 63/399,956, filed on Aug. 22, 2022.

(51) Int. Cl. A61K 35/16; A61K 9/00; A61K 35/35; A61P 13/10

(52) U.S. Cl. CPC A61K 35/16; A61K 9/0019; A61K 35/35; A61P 13/10

(57) ABSTRACT

Provided herein are methods for treating overactive bladder (OAB) and stress urinary incontinence (SUI). The method for treating OAB includes administering a therapeutically effective amount of plasma adjacent to a subject's posterior bladder wall. The method for treating SUI includes administering a therapeutically effective amount of adipose cells mixed with plasma adjacent to a subject's urethra. Also provided herein are kits for treating OAB and SUI according to the methods.

### CROSS-REFERENCE TO RELATED APPLICATION(S)

The present application claims priority under 35 U.S.C. § 119(e) to U.S. Provisional Patent Application Nos. 63/399,956, filed Aug. 22, 2022, which application is incorporated herein by reference in its entirety.

### BACKGROUND OF THE INVENTION

In the medical field, stress urinary incontinence (SUI) is defined as leakage of urine during any activity that causes an increase in abdominal pressure. Patients complain of leakage of urine in connection with coughing, sneezing, laughing, running, other modalities of exercise, and sexual activities. Up to 35% of women have SUI, which contributes to social health consequences (e.g., isolation, depression, etc.). Of the treatments for SUI, the most-successful procedure (approximately 80-90%) involves a permanent polypropylene mesh, which has a serious stigma to it (and is no longer approved by the FDA for the transvaginal repair of pelvic organ prolapse (POP)). This stigma gives patients angst in choosing this type of treatment.

Overactive bladder (OAB) or urinary urgency, frequency, or nocturia, is defined as a sudden need to urinate with or without leakage. OAB is more common in women than in men. Depending on age and gender, the prevalence rates have been almost half of populations studied. Although there are treatments for this condition, the options can be time consuming, invasive and/or have undesirable side effects.

Accordingly, there is a need in the art for articles and methods that improve on existing treatments for SUI and OAB. The present invention addresses this need.

### SUMMARY

In one aspect, provided herein is a method for treating overactive bladder, the method including administering a therapeutically effective amount of plasma adjacent to a subject's posterior bladder wall. In some embodiments, the plasma comprises one or more selected from the group consisting of: platelet-rich plasma (PRP) and platelet-poor plasma (PPP). In some embodiments, the plasma is administered via a trans-vesical injection. In some embodiments, the plasma is autologous to the subject. In some embodiments, the method further includes mixing the plasma with adipose cells.

In another aspect, provided herein is a kit for treating overactive bladder, the kit including a syringe adapted and configured for drawing blood from a subject; a centrifuge adapted and configured for separating the blood into constituent parts including at least plasma; and a trans-vesical needle adapted and configured for injection of the plasma adjacent to the subject's bladder. In some embodiments, the kit further includes instructions for performing the method according to one or more of the embodiments disclosed herein.

In another aspect, provided herein is a method for treating stress urinary incontinence, the method including administering a therapeutically effective amount of adipose cells mixed with plasma adjacent to a subject's urethra. In some embodiments, the plasma comprises one or more selected from the group consisting of platelet-rich plasma (PRP) and platelet-poor plasma (PPP). In some embodiments, the plasma is autologous to the subject. In some embodiments, the adipose cells are autologous to the subject. In some embodiments, the adipose cells are administered via a trans-urethral injection. In some embodiments, the adipose cells include a reduced average diameter. In some embodiments, the adipose cells are micronized. In some embodiments, the adipose cells are atomized.

In another aspect, provided herein is a kit for treating stress urinary incontinence, the kit including a fat harvester adapted and configured for harvesting adipose cells from a subject; and a trans-urethral needle adapted and configured for injection of the adipose cells adjacent to the subject's urethra. In some embodiments, the kit further includes instructions for performing the method according to one or more of the embodiments disclosed herein. In some embodiments, the kit further includes a centrifuge adapted and configured for separating the adipose cells from serum.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a method for treating overactive bladder (OAB) according to an embodiment of the invention.

FIG. 2 depicts a kit for treating overactive bladder (OAB) according to an embodiment of the invention.

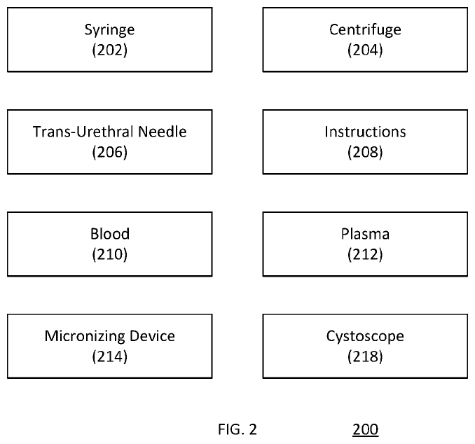

FIG. 3 depicts a method for treating stress urinary incontinence (SUI) according to an embodiment of the invention.

FIG. 4 depicts a kit for treating stress urinary incontinence (SUI) according to an embodiment of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

The terms “proximal” and “distal” can refer to the position of a portion of a device relative to the remainder of the device or the opposing end as it appears in the drawing. The proximal end can be used to refer to the end manipulated by the user. The distal end can be used to refer to the end of the device that is inserted and advanced and is furthest away from the user. As will be appreciated by those skilled in the art, the use of proximal and distal could change in another context, e.g., the anatomical context in which proximal and distal use the patient as reference, or where the entry point is distal from the user.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Provided herein are articles and methods for treating stress urinary incontinence (SUI) and/or overactive bladder (OAB). In some embodiments, the methods for treating SUI and/or OAB include preparing and/or administering a composition for periurethral bulking. In some embodiments, a method of preparing a composition for treating SUI and/or OAB includes providing at least one of platelet rich plasma (PRP) from a subject, platelet-poor plasma (PPP), and fat cells from a subject, and forming the composition from at least one of the PRP, the PPP, and the fat cells. The PRP may include, but is not limited to, leukocyte-rich PRP (LPRP), leukocyte-reduced PRP (P-PRP; leukocyte-reduced or pure PRP), leukocyte platelet-rich fibrin, or pure platelet-rich fibrin. The subject from which the PRP, PPP, and/or fat cells are provided may be the same subject or different subjects, and may be the subject to be treated or a different subject.

In some embodiments, providing the PRP and/or PPP includes obtaining or withdrawing a blood specimen from the subject and centrifuging the blood specimen to separate PRP from red blood cells and platelet poor plasma. In some embodiments, centrifuging the blood specimen includes first centrifuging in a soft spin to produce a platelet rich layer, a superficial buffy coat layer, and a red blood cell layer. The soft spin includes any suitable rate and time to separate the platelet rich layer and superficial buffy coat layer from the red blood cell layer, such as, but not limited to, 200g for 20 minutes. After soft spinning, the platelet rich layer and the superficial buffy coat layer are extracted (without the red blood cell layer) and centrifuged at a higher spin to form a PPP layer and layer including pellets of platelets. The higher spin includes any suitable rate and time to form the PPP layer and/or layer including pellets of platelets, such as, but not limited to, 2500 g for 15 minutes. Following the higher spin, the PPP is removed and disposed of or stored for later use. Additionally or alternatively, the remaining pellets are homogenized to form the final PRP product.

In some embodiments, providing the fat cells includes harvesting fat cells from the subject. In some embodiments, harvesting the fat cells includes locating a site for fat harvesting, and slowly aspirating fat from the site with gentle pressure from syringe withdrawal. Any suitable syringe may be used, such as, but not limited to, a luer lock syringe and blunt cannula disposable fat harvester. In some embodiments, the syringe withdrawal provides extraction with damaging adipocytes, which is possible with machine extraction. In some embodiments, after aspirating, the syringe is removed and turned upside down to bring fat to the surface, then the serum is expelled while the fat is retained in the syringe. Additionally or alternatively, the fat may be centrifuged after aspirating to separate fat from other components. In some embodiments, the fat can be harvested while the blood is being centrifuged.

As will be appreciated by those skilled in the art, the method of preparing the composition may differ depending upon the disorder being treated. In some embodiments, preparing the composition for treating OAB includes separating one or more components of plasma. For example, the method of preparing the composition for treating OAB may include separating PRP, PPP, or PRP and PPP into different containers. In some embodiments, preparing the composition for treating OAB further includes mixing adipose cells with one or more of the components of plasma. Alternatively, in some embodiments, preparing the composition for treating SUI includes mixing adipose cells with plasma (e.g., PRP and/or PPP). For example, in some embodiments, preparing the composition for treating SUI includes homogenously mixing adipose cells and PRP. The homogenous mixture may include any suitable volumetric fat: PRP ratio, such as, but not limited to, between about 2:1 and about 4:1, about 2:1, about 3:1, about 4:1, or any combination, sub-combination, range, or sub-range thereof. Preparing a homogenous mixture of fat and PRP includes mixing the fat and PRP in any suitable manner. In some embodiments, for example, mixing the fat and PRP includes sieving through two syringes, back and forth, until homogenous mixture is created. In some embodiments, mixing the fat and PRP includes mixing with a three way luer lock connector.

In some embodiments, preparing the composition includes reducing the maximum cross-sectional dimension of the fat particles. In some embodiments, reducing the maximum cross-sectional dimension of the fat particles includes micronizing, atomizing, or otherwise reducing the size of the fat cells. The cross-sectional dimension of the fat particles may be reduced directly (i.e., in a stand-alone composition of fat cells or prior to mixing with plasma) and/or after mixing with the plasma. For example, in some embodiments, the fat cells are micronized or atomized before being mixed with the PRP. Additionally or alternatively, the fat cells or mixture of fat cell and plasma may be sieved to remove larger particles.

Also provided herein are compositions for treating OAB and/or SUI. The composition may include any composition according to one or more of the embodiments disclosed herein. For example, in some embodiments, the composition includes one or more components of blood plasma (e.g., PRP and/or PPP) for use in treating OAB. In some embodiments, the composition for use in treating OAB includes plasma mixed with one or more components of adipose cells. In some embodiments, the composition for treating SUI includes adipose cells. In some embodiments, the adipose cells are reduced in diameter, such as through micronizing and/or atomizing. Additionally or alternatively, in some embodiments, the composition for treating SUI includes adipose cells mixed with one or more components of blood plasma. For example, in some embodiments, the composition for treating SUI includes a homogenous mixture of adipose cells and PRP.

Further provided herein are methods of treating a subject for OAB and/or SUI. The methods include administering one or more of the compositions disclosed herein to a subject in need thereof. As will be appreciated by those skilled in the art, the method of treating the subject for OAB and/or SUI may include the methods of preparing the composition disclosed herein, or the composition may be provided in ready-to-use form for treatment.

The method of treating a subject for OAB includes administering a therapeutically effective amount of plasma to the subject. In some embodiments, the method of treating a subject for OAB includes administering separate therapeutically effective amounts of PRP and/or PPP to the subject. Additionally or alternatively, in some embodiments, one or more of the plasma components (e.g., PRP and/or PPP) may be mixed with one or more components of adipose cells. The plasma may be administered by any suitable mechanism (e.g., trans-vesical injection) and may be injected in any suitable location for treating OAB. Suitable locations for treating OAB include, but are not limited to, adjacent to a subject's posterior bladder wall (e.g., at the trigone and posterior bladder wall depending on the bladder capacity). The injection for OAB can be terminated when blebs can be first visualized (e.g., as detected using direct cystoscope visualization or imaging modalities such as ultrasound). In some embodiments, the radial spacing of the needles promotes uniform narrowing of urethra due to the bulking material injected. The provider can wait a defined duration (e.g., about 30 seconds) to allow for equalization of pressure.

The method of treating a subject for SUI includes administering a therapeutically effective amount of adipose cells mixed with plasma to the subject. In some embodiments, for example, the method of treating a subject for SUI includes administering a homogenous mixture of adipose cells and PRP (or PPP) to the subject. In some embodiments, a separate amount of PPP (or PRP) may be administered to the subject along with the homogenous mixture of adipose cells and PRP (or PPP). The mixture of adipose cells and plasma may be administered by any suitable mechanism (e.g., trans-urethral injection) and may be injected in any suitable location for treating SUI. Suitable locations for treating SUI include, but are not limited to, approximately 1-1.5 cm at midurethra, and at bladder neck, approximately 3-5 cm from the external urethral orifice, depending on the total length of the urethra. The injection for SUI can be terminated when blebs extend to about the middle of the lumen of the urethra (e.g., as detected using direct cystoscope visualization or imaging modalities such as ultrasound). In some embodiments, the radial spacing of the needles promotes uniform narrowing of urethra due to the bulking material injected. The provider can wait a defined duration (e.g., about 30 seconds) to allow for equalization of pressure.

Still further provided herein are kits for treating OAB and/or SUI. In some embodiments, a kit for treating overactive bladder includes a syringe adapted and configured for drawing blood from a subject; a centrifuge adapted and configured for separating the blood into constituent parts including at least plasma; and a trans-vesical needle adapted and configured for injection of the plasma adjacent to the subject's bladder. In some embodiments, the kit further includes instructions for performing one or more of the methods disclosed herein. In some embodiments, a kit for treating stress urinary incontinence includes a fat harvester adapted and configured for harvesting adipose cells from a subject; and a trans-urethral needle adapted and configured for injection of the adipose cells adjacent to the subject's urethra. In some embodiments, the kit further includes a centrifuge adapted and configured for separating the adipose cells from serum. In some embodiments, the kit further includes instructions for performing one or more of the methods disclosed herein.

In some embodiments, the articles and methods disclosed herein facilitate treatment of stress urinary incontinence (SUI) and/or overactive bladder (OAB) without the use of a permanent mesh. As described herein, the conditions for preparing the compositions and methods of administering the same may differ between conditions. In this regard, below are examples relating to the preparation and administration of the composition in both conditions.

### Example Methods for Preparing Compositions to Treat SUI and OAB

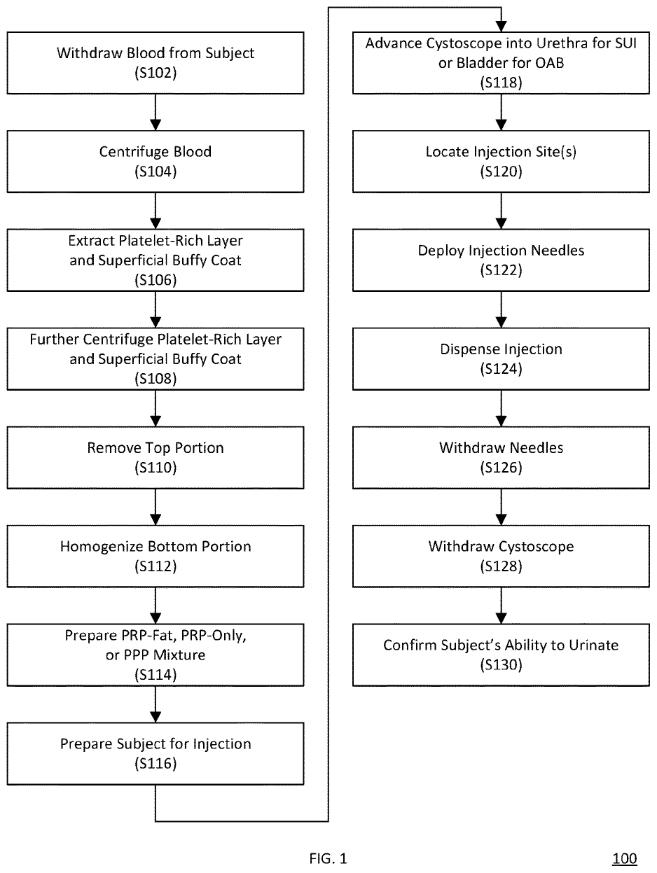

In some embodiments, as illustrated in FIG. 1, the methods disclosed herein include a method 100 for treating stress urinary incontinence (SUI) and/or overactive bladder (OAB). The first portion of the method show in FIG. 1 related to preparation of a composition to treat SUI, according to an embodiment of the disclosure.

In step S102, blood 210 is withdrawn from a subject. The blood 210 can be withdrawn using techniques such as venipuncture and devices such as a butterfly needle and syringe 202 (e.g., a 20 ml syringe with a Luer tip). The volume drawn can reflect the desired volume of plasma to be utilized later in the method 100. For example, a 30 mL venous blood draw can produce between about 3 mL and about 5 mL of platelet-rich plasma (PRP). In some embodiments, the volume is about 60 ml. In some embodiments, the blood 210 is drawn using a tube containing an anti-coagulant such as acid citrate dextrose (ACD), sodium citrate, citrate dextrose, and the like. Alternatively, an anticoagulant can be added to the tube after the blood 210 is drawn. In either embodiment, the vessel can be inverted to promote mixing of the anticoagulant with the blood 210 after drawing.

In step S104, the whole blood is centrifuged to separate components such as plasma. For example, whole blood can be centrifuged in “soft spin” at about 200 g for about 20 minutes to produce three layers: platelet-rich layer (top); superficial buffy coat (middle), and red blood cells (bottom). Suitable centrifuges 204 include the TRUPRP MAGELLAN® centrifuge available from Timeless Age Medical of Hallandale, Florida. Next, in step S106, the platelet-rich layer (top) and superficial buffy coat (middle) can be extracted, and then further centrifuged in step S108. In one embodiment, the platelet-rich layer (top) and superficial buffy coat (middle) are further centrifuged at about 2,500 g for about 15 minutes.

In steps S110 and S112, plasma 212 can be separated. For example, in step S110, a top portion following step S108 (e.g., the upper 2/3 constituting platelet-poor plasma(PPP)) can be removed. In some embodiments, such as for treatment of OAB, the PPP can be saved for administration to the subject. Alternatively, the PPP can be disposed of and, in step S112, the bottom portion (e.g., pellets of platelet-rich plasma) can be homogenized. The platelet-rich plasma can be leukocyte-rich PRP (LPRP), leukocyte-reduced PRP (P-PRP; leukocyte-reduced or pure PRP), leukocyte platelet-rich fibrin, or pure platelet-rich fibrin.

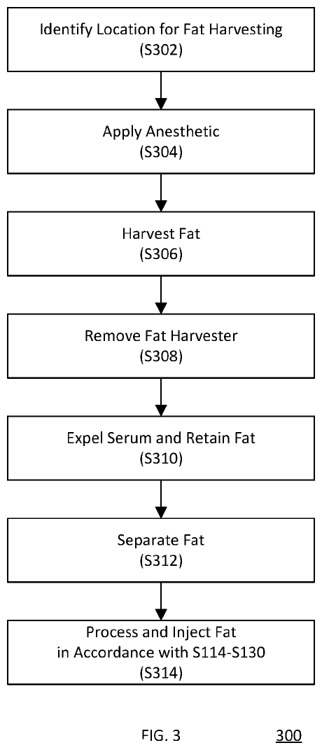

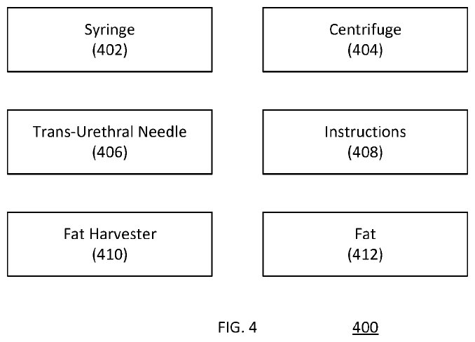

Turning to FIG. 3 and FIG. 4, in some embodiments, a method 300 is provided for harvesting fat cells from a subject. These fat cells may be administered directly or mixed with the PRP to form the PRP-Fat mixture of step S114. Accordingly, method 300 may be performed simultaneously or sequentially with method 100.

In step S302, a location for fat harvesting (e.g., in the hip region) is located and cleaned (e.g., with isopropyl alcohol, iodine, and the like). In step S304, an anesthetic (e.g., lidocaine) is applied to the region. In step S306, a fat harvester 410 is used to harvest fat from the region.

Suitable fat harvesters include cannulas having side ports such as the Carraway harvester available from Black Tie Medical, Inc. dba Tulip Medical Products of San Diego, California. In other embodiments, the fat can be harvested using liposuction tools.

In step S308, the fat harvester 410 can be removed from the region. In step S310, serum is expelled from the syringe and fat is retained. This can be performed by advancing the plunger of a syringe 402 coupled to the cannula.

In step S312, the fat 412 can be separated by centrifugation or decantation. The plunger can be removed from the syringe prior to centrifuging. Centrifuging can be performed for about three minutes. In some embodiments, a middle layer is selected to obtain the purest composition of fat. Suitable centrifuges 404 include the TRUPRP MAGELLAN® centrifuge available from Timeless Age Medical of Hallandale, Florida.

In step S314, the fat 412 can be processed and/or injected in accordance with any, all, or a subset of steps S114-S130, which are not repeated here for efficiency.

In step S114, a PRP-fat mixture can be prepared, e.g., by sieving back-and-forth through two syringes until a homogenous mixture is created. In some embodiments, the PRP-fat mixture has a volumetric ratio of about 1-part PRP to 2-3-parts fat. This can be achieved using a micronizing device 214 such as TULIP® microkit available from Black Tie Medical, Inc. dba Tulip Medical Products of San Diego, California. The micronizing device can reduce the maximum cross-sectional dimension of the fat particles to about 1.2 mm, e.g., by first sieving to about 2.4 mm, then with a smaller sieve to reduce to 1.2 mm.

### Example Methods for Treating SUI and OAB

In step S116, the subject can be prepared for injection. For example, the subject can be draped and assume a lithotomy position: on back, knees at 70-90 degree angle from hips. The subject's skin can be cleaned (e.g., with isopropyl alcohol, iodine, and the like). Plain or lidocaine lubricating gel can be injected into urethra about 5-10 minutes before beginning cystoscopy.

In step S118, a cystoscope 218 (e.g., the cystoscope as described in 63,399,897) is advanced into the subject's urethra for SUI (or bladder for OAB). In step S120, injection sites are located within the urethra for SUI (or posterior bladder wall for OAB). Exemplary injection sites for SUI include approximately 1-1.5 cm at midurethra, and at bladder neck, approximately 3-5 cm from the external urethral orifice, depending on the total length of the urethra. Exemplary injection sites include at the trigone and posterior bladder wall depending on the bladder capacity. The location of injection sites can be aided by depth-guide markings on the outside of the cystoscope, using imaging, and the like.

In step S122, after locating the injection sites, injection needles 206 can be deployed from the cystoscope into and/or through the urethra for SUI (or bladder for OAB). In some embodiments, the injection needles have shape memory or are otherwise curved to deploy laterally from the cystoscope. In some embodiments, the needles are configured to deploy to a depth (measured from an outer surface of the urethra) of a least about 0.5 cm. In some embodiments, multiple injections (e.g., 3) are made at radially (e.g., 120-spaced) locations.

In step S124, an injection is dispensed, e.g., by depressing a plunger. The injection may include any composition disclosed herein depending upon the condition being treated. For example, for treatment of SUI the composition may include PRP or a PRP-fat mixture, while for OAB the composition may include PRP, a PRP-fat mixture, and/or PPP. The injection for SUI can be terminated when blebs extend to about the middle of the lumen of the urethra (e.g., as detected using direct cystoscope visualization or imaging modalities such as ultrasound). The injection for OAB can be terminated when blebs can be first visualized (e.g., as detected using direct cystoscope visualization or imaging modalities such as ultrasound). The radial spacing of the needles can promote uniform narrowing of urethra due to the bulking material injected. The provider can wait a defined duration (e.g., about 30 seconds) to allow for equalization of pressure.

In step S126, the needles 206 can be withdrawn into the cystoscope. In step S128, the cystoscope 218 can be withdrawn. In step S130, the provider can confirm the patient's ability to urinate to ensure that the urethra was not over-bulked and/or that the bladder remains intact.

### Kits for Treating SUI and OAB

A kit for treating SUI may include: (1) Alcohol, syringes and butterfly needles to take blood from the patient; (2) Lidocaine 1% with epinephrine premeasured; (3) Carraway disposable fat harvester; (4) 20 ml luer lock syringe; (5) A kit to decrease the size of the fatwith to micronize the fat to 2.4 mm, then 1.2 mm; (6) 2×10 ml syringes to place the solution [PRP+fat]; and (7) A needle device that would be utilized through cystoscopy to inject at 1,4, 7,11:00, 1-1.5 cm deep at the midurethra and at the bladder neck.

A kit for treating OAB may include (1) Alcohol, syringes and butterfly needles to take blood from the patient; (2) 4×5 ml syringes to place the PRP and PPP; (3) A needle device that would be utilized through cystoscopy; (4) 25-gauge 3 inch spinal needle.

### WORKING EXAMPLE

Currently ten patients have had the periurethral bulking procedure for SUI with the PRP and fat harvest using a 22-fr spinal needle. Five of the ten patients (50%) had complete dryness up to 12 months out, while four of the ten patients were between 60-80% dry up to 12 months out. One patient was only dry for 2 weeks then she had a midurethral sling. Two additional patients were treated using atomized adipose tissue and they are completely dry 3 months out. There were no adverse events and the 90% of patients were happy with their results.

There are currently five patients who have had an injection procedure through the anterior vaginal wall close to the posterior bladder wall for overactive bladder. All five patients have had at least 50% improvement of their symptoms six months to one year after their procedure.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method for treating overactive bladder, the method comprising:
administering a therapeutically effective amount of plasma adjacent to a subject's posterior bladder wall.

2. The method of claim 1, wherein the plasma comprises one or more selected from the group consisting of: platelet-rich plasma (PRP) and platelet-poor plasma (PPP).

3. The method of claim 1, wherein the plasma is administered via a trans-vesical injection.

4. The method of claim 1, wherein the plasma is autologous to the subject.

5. The method of claim 1, further comprising mixing the plasma with adipose cells.

6. A kit for treating overactive bladder, the kit comprising:
a syringe adapted and configured for drawing blood from a subject;
a centrifuge adapted and configured for separating the blood into constituent parts including at least plasma; and
a trans-vesical needle adapted and configured for injection of the plasma adjacent to the subject's bladder.

7. The kit of claim 5, further comprising:
instructions for performing the method of claim 1.

8. A method for treating stress urinary incontinence, the method comprising:
administering a therapeutically effective amount of adipose cells mixed with plasma adjacent to a subject's urethra.

9. The method of claim 8, wherein the plasma comprises one or more selected from the group consisting of platelet-rich plasma (PRP) and platelet-poor plasma (PPP).

10. The method of claim 9, wherein the plasma is autologous to the subject.

11. The method of claim 8, wherein the adipose cells are autologous to the subject.

12. The method of claim 8, wherein the mixture of adipose cells and plasma is administered via a trans-urethral injection.

13. The method of claim 8, wherein the adipose cells include a reduced average diameter.

14. The method of claim 13, wherein the adipose cells are micronized.

15. The method of claim 13, wherein the adipose cells are atomized.

16. A kit for treating stress urinary incontinence, the kit comprising:
a fat harvester adapted and configured for harvesting adipose cells from a subject; and
a trans-urethral needle adapted and configured for injection of the adipose cells adjacent to the subject's urethra.

17. The kit of claim 16, further comprising:
instructions for performing the method of claim 8.

18. The kit of claim 16, further comprising:
a centrifuge adapted and configured for separating the adipose cells from serum.
